(19) United States
(12) Patent Application Publication
(10) Pub. No.: US 2017/0303863 A1
(43) Pub. Date: Oct. 26, 2017

(54) OBJECT INFORMATION OBTAINING APPARATUS AND CONTROL METHOD THEREOF

(71) Applicant: CANON KABUSHIKI KAISHA, Tokyo (JP)

(72) Inventors: Toshinobu Tokita, Yokohama-shi (JP); Seiji Yoshimura, Chigasaki-shi (JP); Ayumi Kabata, Kawasaki-shi (JP); Katsuya Oikawa, Tokyo (JP)

(21) Appl. No.: 15/485,342

(22) Filed: Apr. 12, 2017

(30) Foreign Application Priority Data
Apr. 20, 2016 (JP) 2016-084736

(51) Int. Cl. A61B 5/00; A61B 8/08; A61B 8/00; A61B 8/14; A61B 8/06; A61B 5/026

(52) U.S. Cl. CPC A61B 5/7285; A61B 8/543; A61B 8/5223; A61B 8/46; A61B 8/4416; A61B 5/0035; A61B 8/06; A61B 5/7282; A61B 5/026; A61B 5/0095; A61B 8/14

(57) ABSTRACT

An object information obtaining apparatus is used which includes: an irradiator; a transmitting element transmitting an ultrasound wave; a receiving element receiving an acoustic wave; a signal processor; an information processor; and a controller, wherein the signal processor generates a photoacoustic signal and an ultrasound echo signal, the information processor obtains photoacoustic characteristic information and ultrasound characteristic information including information related to blood flow, and the controller controls, in accordance with the blood flow, at least one of light irradiation, generation of the photoacoustic signal, and obtaining of the photoacoustic characteristic information.

### BACKGROUND OF THE INVENTION

### Field of the Invention

The present invention relates to an object information obtaining apparatus and a control method thereof.

### Description of the Related Art

Photoacoustic tomography (PAT) is attracting attention as a method of specifically imaging angiogenesis which occurs due to cancer. PAT is a system involving illuminating an object with illuminating light (near infrared light), receiving a photoacoustic wave emitted from inside the object with an ultrasound probe, and converting the photoacoustic wave into an image.

FIG. 7 shows a schematic diagram of a hand-held photoacoustic apparatus described in S. A. Ermilov et al., Development of laser optoacoustic and ultrasound imaging system for breast cancer utilizing handheld array probes, Photons Plus Ultrasound: Imaging and Sensing 2009, Proc. of SPIE vol. 7177, 2009. A photoacoustic probe 104 is configured such that a receiver 106 for receiving a photoacoustic wave is sandwiched and fixed by an illumination optical system 105 including an exit end of a bundled fibers 3. Illuminating light from a light source 101 is incident to an incident end 103a of the bundled fibers 103. When an object is irradiated with the illuminating light, a photoacoustic wave is emitted from the object. The receiver 106 receives the photoacoustic wave, converts the photoacoustic wave into an electrical signal, and transmits the electrical signal to a processing apparatus 107 of an ultrasound apparatus. The processing apparatus 107 performs amplification, digitization, and image reconstruction on the electrical signal to generate image data. A monitor 108 displays a photoacoustic image.

S. A. Ermilov et al., Development of laser optoacoustic and ultrasound imaging system for breast cancer utilizing handheld array probes, Photons Plus Ultrasound: Imaging and Sensing 2009, Proc. of SPIE vol. 7177, 2009 describes clinical research related to breast cancer. A location of breast cancer is identified by an ultrasound scan using an ultrasound probe (the receiver 106) and an ultrasound image of the identified region is recorded. Subsequently, a measurement is performed by switching to a real-time photoacoustic mode.

Non Patent Literature 1: S. A. Ermilov et al., Development of laser optoacoustic and ultrasound imaging system for breast cancer utilizing handheld array probes, Photons Plus Ultrasound: Imaging and Sensing 2009, Proc. of SPIE vol. 7177, 2009

### SUMMARY OF THE INVENTION

However, the related art described above has the following problems. Photoacoustic measurement is a technique in which illuminating light is supplied and an acoustic wave emitted from a light absorber is received. Therefore, the deeper a depth from a surface of an object to the light absorber, the weaker a photoacoustic signal. When the light absorber is blood (in particular, blood inside a newly generated blood vessel due to cancer), the deeper the depth of cancer from skin (a surface), the more difficult it is to receive a photoacoustic signal. In this case, a method of inserting a probe into the object to reduce a distance from the surface of the object to the light absorber is conceivable. However, pushing the probe into the object may result in reducing a blood flow rate inside a living organism or stopping blood flow. As a result, since blood constituting the light absorber decreases, the photoacoustic signal may weaken and cause a decline in contrast.

In addition, in PAT, by obtaining a photoacoustic signal for each wavelength of illuminating light with a plurality of wavelengths, functional information of blood such as oxygen saturation can be measured. Even in this case, pushing a probe into a surface of an object may reduce a blood flow rate or stop blood flow and may result in changing functional information represented by oxygen saturation and a total hemoglobin amount. As described above, accuracy of acquirable information on an object may decline under reduced blood flow.

The present invention has been made in consideration of the problems described above. An object of the present invention is to suppress an effect of a decline in a blood flow rate in photoacoustic measurement.

The present invention provides an object information obtaining apparatus, comprising:

an irradiator configured to irradiate an object with light output from a light source;

a transmitting element configured to transmit a transmission ultrasound wave;

a receiving element configured to receive a reception acoustic wave and output an electrical signal;

a signal processor;

an information processor; and

a controller, wherein

the signal processor is configured to generate a photoacoustic signal from the electrical signal derived from a photoacoustic wave generated inside the object when the object is irradiated with the light and to generate an ultrasound echo signal from the electrical signal derived from an echo wave generated when the transmission ultrasound wave is reflected inside the object,

the information processor is configured to obtain photoacoustic characteristic information of the object using the photoacoustic signal and to obtain ultrasound characteristic information of the object including information related to blood flow of the object using the ultrasound echo signal, and

the controller is configured to, in accordance with the information related to the blood flow, perform control of at least one of irradiation of the light, generation of the photoacoustic signal, and obtaining of the photoacoustic characteristic information.

The present invention also provides a control method of an object information obtaining apparatus including an irradiator which irradiates an object with light output from a light source, a transmitting element which transmits a transmission ultrasound wave, a receiving element which receives a reception acoustic wave and which outputs an electrical signal, a signal processor, an information processor, and a controller, wherein the control method of an object information obtaining apparatus comprises:

causing the signal processor to generate a photoacoustic signal from the electrical signal derived from a photoacoustic wave generated inside the object when the object is irradiated with the light and generate an ultrasound echo signal from the electrical signal derived from an echo wave generated when the transmission ultrasound wave is reflected inside the object;

causing the information processor to obtain photoacoustic characteristic information of the object using the photoacoustic signal and obtain ultrasound characteristic information of the object including information related to blood flow of the object using the ultrasound echo signal; and

causing the controller to perform control of, in accordance with the information related to the blood flow, at least one of irradiation of the light, generation of the photoacoustic signal, and obtaining of the photoacoustic characteristic information.

According to the present invention, an effect of a decline in a blood flow rate in photoacoustic measurement can be suppressed.

### BRIEF DESCRIPTION OF THE DRAWINGS

FIG. 1 is a diagram illustrating a configuration of a photoacoustic apparatus;

FIG. 2 is a timing chart illustrating photoacoustic obtaining and Doppler obtaining;

FIG. 3A is a flow chart illustrating a control method according to a first embodiment;

FIG. 3B is another flow chart illustrating a control method according to the first embodiment;

FIG. 4 is a flow chart illustrating a control method according to a second embodiment;

FIG. 5A is a flow chart illustrating a control method according to a third embodiment;

FIG. 5B is another flow chart illustrating a control method according to the third embodiment;

FIG. 6 is another timing chart illustrating photoacoustic obtaining and Doppler obtaining; and

FIG. 7 is a diagram illustrating a configuration of a photoacoustic apparatus of background art.

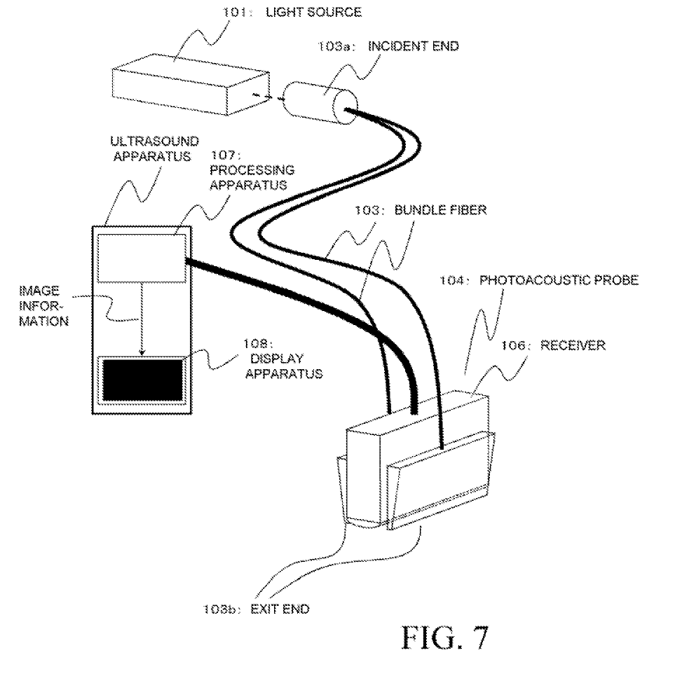

Further features of the present invention will become apparent from the following description of exemplary embodiments with reference to the attached drawings.

### DESCRIPTION OF THE EMBODIMENTS

Hereinafter, preferred embodiments of the present invention will be described with reference to the drawings. However, it is to be understood that dimensions, materials, shapes, relative arrangements, and the like of components described below are intended to be changed as deemed appropriate in accordance with configurations and various conditions of apparatuses to which the present invention is to be applied. Therefore, the scope of the present invention is not intended to be limited to the description presented below.

The present invention relates to a technique for detecting an acoustic wave propagating from an object and generating and obtaining characteristic information of the inside of the object. Accordingly, the present invention can be considered an object information obtaining apparatus or a control method thereof, or an object information obtaining method and a signal processing method. The present invention can also be considered a program that causes an information processing apparatus including hardware resources such as a CPU and a memory to execute these methods or a storage medium storing the program.

The object information obtaining apparatus according to the present invention includes an apparatus utilizing a photoacoustic effect in which an acoustic wave generated inside an object by irradiating the object with light (an electromagnetic wave) is received and characteristic information of the object is obtained as image data. In this case, characteristic information refers to information on a characteristic value corresponding to each of a plurality of positions inside the object which is generated using a received signal obtained by receiving a photoacoustic wave.

Characteristic information (photoacoustic characteristic information) derived from an electrical signal (a photoacoustic signal) obtained by photoacoustic measurement is a value reflecting an absorption rate of optical energy. For example, characteristic information includes a generation source of acoustic waves generated by light irradiation, initial sound pressure inside an object, an optical energy absorption density or an absorption coefficient derived from initial sound pressure, and a concentration of substances constituting tissue. In addition, a distribution of oxygen saturation can be calculated by obtaining a concentration of oxygenated hemoglobin and a concentration of reduced hemoglobin as concentrations of substances. Furthermore, a glucose concentration, a collagen concentration, a melanin concentration, a volume fraction of fat or water, and the like can also be obtained.

The object information obtaining apparatus according to the present invention includes an apparatus using ultrasound echo technology in which an ultrasound wave is transmitted to an object, a reflected wave (an echo wave) that is reflected inside the object is received, and object information is obtained as image data. Characteristic information (ultrasound characteristic information) derived from an electrical signal (an ultrasound echo signal) obtained by an ultrasound echo apparatus is information reflecting a difference in acoustic impedances among tissues inside an object.

Particularly, in the present invention, ultrasound characteristic information includes information related to blood flow which is obtained based on a principle of a Doppler method. Specifically, ultrasound characteristic information includes Doppler information reflecting a blood flow speed or a blood flow rate which is obtained based on a frequency of a transmission ultrasound wave and a frequency of a reception acoustic wave when the transmission ultrasound wave is reflected by a living organism.

A two-dimensional or three-dimensional characteristic information distribution is obtained based on characteristic information at each position in the object. Distribution data may be generated as image data. Characteristic information may be obtained as distribution information of respective positions inside the object instead of as numerical data. In other words, distribution information such as a distribution of initial sound pressure, a distribution of energy absorption density, a distribution of absorption coefficients, and a distribution of oxygen saturation may be obtained. In addition, an acoustic impedance distribution, distribution information representing blood flow, and the like may be generated.

An acoustic wave as referred to in the present invention is typically an ultrasound wave and includes an elastic wave which is also referred to as a sonic wave or an acoustic wave. An electrical signal converted from an acoustic wave by a probe or the like is also referred to as an acoustic signal. However, descriptions of an ultrasound wave and an acoustic wave in the present specification are not intended to limit a wavelength of the elastic waves. An acoustic wave generated by a photoacoustic effect is referred to as a photoacoustic wave or an optical ultrasound wave. An electrical signal derived from a photoacoustic wave is also referred to as a photoacoustic signal. In addition, an electrical signal derived from an echo wave generated when a transmission ultrasound wave is reflected by an object is also referred to as an ultrasound echo signal. In the present specification, acoustic waves propagating from an object may be collectively referred to as reception acoustic waves. Reception acoustic waves include a photoacoustic wave and an echo wave.

### First Embodiment

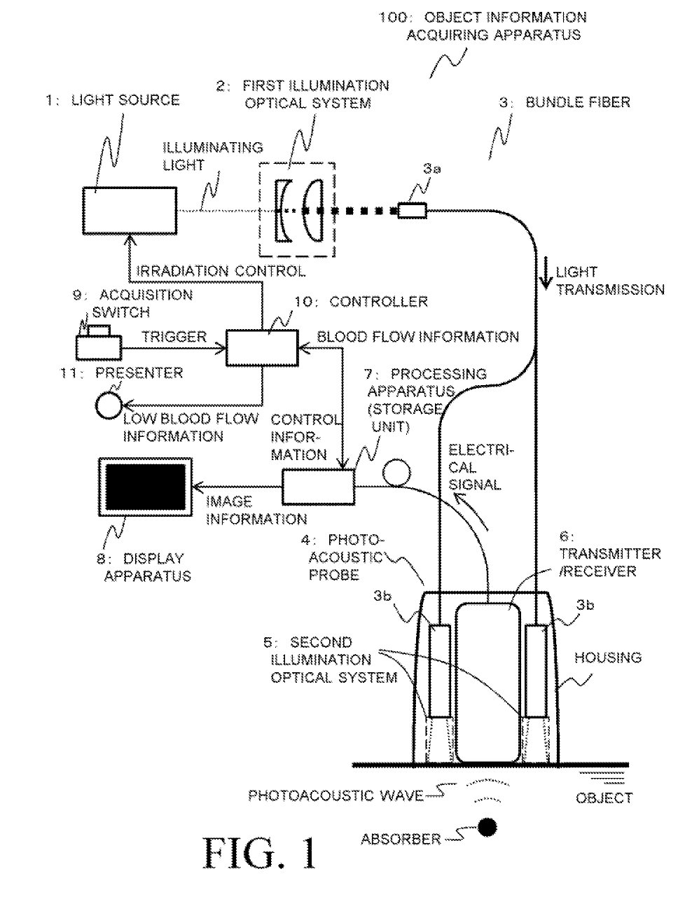

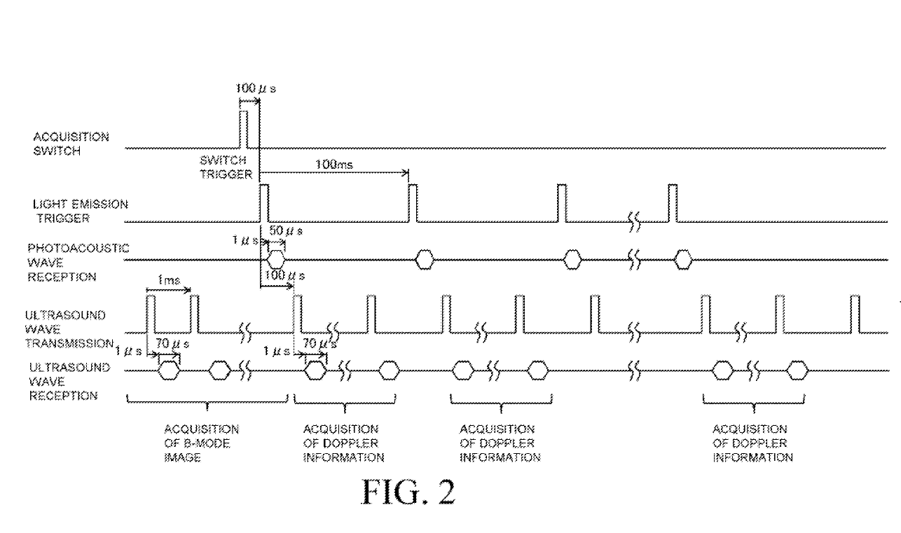

A basic embodiment will be described using FIGS. 1 and 2. FIG. 1 is a schematic diagram of an object information obtaining apparatus 100. FIG. 2 is a timing chart illustrating a flow of signals and information.

### Apparatus Configuration

In FIG. 1, a light source 1 emits illuminating light. A first illumination optical system 2 shapes the illuminating light and causes the illuminating light to enter an incident end 3a of a bundled fibers 3. The bundled fibers 3 transmits the illuminating light to a photoacoustic probe 4 and causes the illuminating light to exit from an exit end 3b of the photoacoustic probe 4. These members correspond to an irradiator according to the present invention.

The photoacoustic probe 4 includes the exit end 3b of illuminating light to an object, a second illumination optical system 5, and a transmitter/receiver 6 which receives a photoacoustic wave emitted from the object. The second illumination optical system 5 is constituted by a prism, a diffuser plate, or the like. The transmitter/receiver 6 converts an acoustic wave (a photoacoustic wave or a reflected echo wave and also referred to as a reception acoustic wave) propagating from the object into an electrical signal and sends the electrical signal to a processing apparatus 7. The transmitter/receiver 6 includes conversion elements capable of converting an electrical signal into an acoustic wave and vice versa such as a piezoelectric element or a CMUT. The conversion elements are favorably arranged in an array from the perspectives of reducing measurement time and enlarging a measurable area. Moreover, the photoacoustic probe 4 is favorably covered by a housing. The second illumination optical system 5 may be configured so as to be attachable and detachable with respect to the photoacoustic probe 4. When the second illumination optical system 5 is not mounted, the photoacoustic probe 4 can be used as an apparatus which exclusively performs ultrasound echo measurement.

The processing apparatus 7 performs amplification, digital conversion, filtering, and the like on an analog electrical signal output from the conversion element. In this regard, the processing apparatus 7 corresponds to a signal processor according to the present invention. The processing apparatus 7 also generates image information indicating characteristic information (photoacoustic characteristic information or ultrasound characteristic information) inside an object based on a digital electrical signal (a photoacoustic signal or an ultrasound echo signal). In this regard, the processing apparatus 7 corresponds to an information processor according to the present invention. The processing apparatus 7 includes a processing circuit constituted by an ASIC, an FPGA, or the like, a CPU, a storage unit (a ROM, a RAM, a hard disk, or the like), and an input UI and can be realized by an information processing apparatus such as a PC which operates in accordance with a program. The processing apparatus 7 may be constructed by combining a plurality of processing circuits and information processing apparatuses. A display apparatus 8 displays an image based on the image information.

When generating photoacoustic characteristic information, the processing apparatus 7 can use various known image reconstruction methods. For example, a phasing addition method, a Fourier transform method, an inverse operation method, a filtered back-projection method, or a universal back-projection method can be used. In addition, any image reconstruction method or a B-mode display method can be used to generate ultrasound characteristic information based on an ultrasound echo signal. When obtaining information related to blood flow such as a flow speed or a change thereof or a blood flow rate from an ultrasound echo signal, known methods such as a continuous wave Doppler method and a pulse Doppler method can be used.

### Operation of Controller

A controller 10 is a controller configured to control light irradiation, generation of a photoacoustic signal, generation of photoacoustic characteristic information, and the like. A processing circuit or an information processing apparatus can be used as the controller 10. A single information processing apparatus may double as the controller 10 and the processing apparatus 7. In the present embodiment, the controller 10 performs functions of a switching unit configured to control measurement modes in response to an operation of an obtaining switch 9 by a user (an operator such as a physician or a technician). In an ultrasound measurement mode used when the obtaining switch 9 is not operated, the transmitter/receiver 6 transmits/receives an ultrasound wave to/from an object. In addition, the processing apparatus 7 obtains a B-mode image and causes the display apparatus 8 to display the image. While the obtaining switch 9 may be provided separately from the photoacoustic probe 4, providing the obtaining switch 9 on the photoacoustic probe 4 enables the user to operate the obtaining switch 9 at hand and is useful for improving user convenience.

On the other hand, as shown in a topmost row of the timing chart in FIG. 2, when the obtaining switch 9 is operated and a switch trigger is raised, a determination of switching from an ultrasound echo mode to a photoacoustic measurement mode is performed. The processing apparatus 7 calculates Doppler information from an ultrasound echo signal, obtains information related to blood flow, and determines whether or not the information satisfies a prescribed condition. Conceivable determination conditions include the presence or absence of a blood flow and whether or not a blood flow rate or a blood flow speed exceeds a prescribed threshold. Alternatively, Doppler information may be obtained a plurality of times and a determination may be made on whether or not a rate or an amount of decline of a blood flow rate or a blood flow speed or, in other words, a value related to an amount of decline of the blood flow rate or the blood flow speed exceeds a prescribed threshold.

When the information related to the blood flow does not satisfy the prescribed condition, the controller 10 presents, through a presenter 11 which is a presenting unit, low blood flow information. The presenter 11 may be configured to appeal to the visual sense by lighting an LED or the like or to appeal to the acoustic sense using an alarm tone or the like. In addition, low blood flow information may be presented through the display apparatus 8 as text information. Furthermore, the technician may be presented with the fact that blood flow is low by displaying Doppler information. Moreover, as shown in a bottommost row in FIG. 2, favorably, the processing apparatus 7 performs condition determination by periodically obtaining Doppler information in parallel with performing photoacoustic measurement.

According to this configuration, the technician can be presented with the fact that an object is in a state of low blood flow. In addition, the technician presented with the information can reduce force applied to the object to avoid a state of low blood flow. As a result, a decline in photoacoustic signal strength can be suppressed and an image with high contrast can be obtained. Furthermore, a favorable image can be obtained in a state of normal blood flow even when measuring functional information such as oxygen saturation using light with a plurality of wavelengths.

FIG. 2 will be further described. Until the obtaining switch 9 is pressed, an ultrasound image (for example, a B-mode image) is displayed in accordance with ultrasound wave transmission/reception. When the obtaining switch 9 is pressed, illuminating light is irradiated from the exit end and, in synchronization with the irradiation of illuminating light, a photoacoustic signal is received. In the example shown in FIG. 2, ultrasound wave transmission/reception is performed between an emission of light and a next emission of light, and Doppler information is calculated from an ultrasound echo signal. In this example, light is emitted at 100 ms intervals (a pulse rate of 10 Hz). Photoacoustic measurement is continued as long as information related to blood flow obtained from the Doppler information satisfies the prescribed condition. When the condition is no longer satisfied, the technician is presented with information to the effect that the blood flow rate has declined.

### Favorable Configuration Example

In FIG. 1, the bundled fibers 3 is branched midway and two exit ends 5 are provided so as to sandwich the transmitter/receiver 6. However, the number of branches is not limited thereto. For example, an unbranched fiber may be brought adjacent to only one surface of the transmitter/receiver 6. In addition, an optical element such as a mirror or a prism may be used in place of the bundled fibers 3. Furthermore, an acoustic matching material (not shown) may be provided in a portion in contact with an object of the transmitter/receiver 6. An acoustic matching material has a characteristic of matching acoustic impedances of a conversion element and an object. For example, a resin material can be used as an acoustic matching material. An acoustic matching material such as an ultrasound gel may also be favorably used.

As the light source 1, a light source emitting near infrared light with a wavelength of around 600 nm to 1100 nm is preferable. For example, a pulse laser such as a Nd:YAG laser or an alexandrite laser is used. In addition, a Ti:sa laser or an OPO laser using Nd:YAG laser light as excitation light may be used. Furthermore, a semiconductor laser may be used. Alternatively, a flash lamp or an LED light source may be used. When a semiconductor laser or an LED light source is used, the light source can be incorporated into the photoacoustic probe 4 and the bundled fibers 3 can be omitted.

Irradiation of illuminating light and reception of photoacoustic waves must be synchronized with each other. In order to do so, a part of the illuminating light emitted from the light source 1 may be branched to be detected by a sensor such as a photo diode and used as a light emission trigger. Alternatively, an emission timing and a reception timing may be synchronized with each other using a signal generator (not shown) such as a pulser.

It has been described above that a B-mode tomographic image is obtained until the obtaining switch 9 is operated. However, transmission/reception may be stopped by a timer in accordance with an MI (mechanical index) value to freeze ultrasound wave transmission/reception. In addition, in a case of an object information obtaining apparatus using a hand-held probe, providing the obtaining switch 9 on a case (not shown) of the photoacoustic probe 4 improves operability. Alternatively, an inputter of an information processing apparatus (for example, a mouse, a keyboard, a touch panel, or the like of a PC) constituting the processing apparatus 7 may be used. An obtaining switch can also be considered a part of an inputter.

The present invention is not limited to a hand-held apparatus. While a case where the photoacoustic probe 4 is brought into contact with an object has been heretofore described, for example, the present invention can also be applied to an apparatus which conducts measurements while the photoacoustic probe 4 performs mechanical scanning in a state where an object is held by a pressing mechanism such as a pair of opposing pressing plates or held by a cup-like holding member. In this case, a liquid such as water or castor oil is preferable as an acoustic matching material.

In the present invention, a conversion element (a receiving element) for receiving reception acoustic waves (an ultrasound echo and a photoacoustic wave) and a conversion element (a transmitting element) for generating transmission ultrasound waves may be provided separately. In addition, an element for transmitting and receiving ultrasound waves (an element which doubles as a transmitting element and a receiving element) and a conversion element (a receiving element) for receiving photoacoustic waves may be provided separately. Furthermore, an ultrasound echo signal may be used only to determine blood flow upon obtaining of photoacoustic characteristic information or ultrasound characteristic information itself may be displayed to the technician. When displaying ultrasound characteristic information, the ultrasound characteristic information is preferably displayed superimposed on a photoacoustic image or displayed side by side with a photoacoustic image. Moreover, Doppler information may be displayed superimposed on or displayed side by side with a photoacoustic image and/or an ultrasound image.

The Doppler method favorably enables blood flow to be displayed such as color Doppler and power Doppler. However, a blood flow measurement method such as PW (pulse wave) can also be used. In this case, an inputter for setting a region of interest (ROI) is favorably provided on the object information obtaining apparatus 100. In addition, an inputter to be used by the technician to set a threshold when determining whether or not a condition related to blood flow is satisfied may be provided. As inputters for the ROI and the threshold, for example, the inputter of the information processing apparatus constituting the processing apparatus 7 can be used. The set ROI and threshold are stored in a storage unit of the information processing apparatus. When designation of a ROI is input, the determination of whether or not the condition related to blood flow is satisfied may be made based on the ROI.

### Process Flow

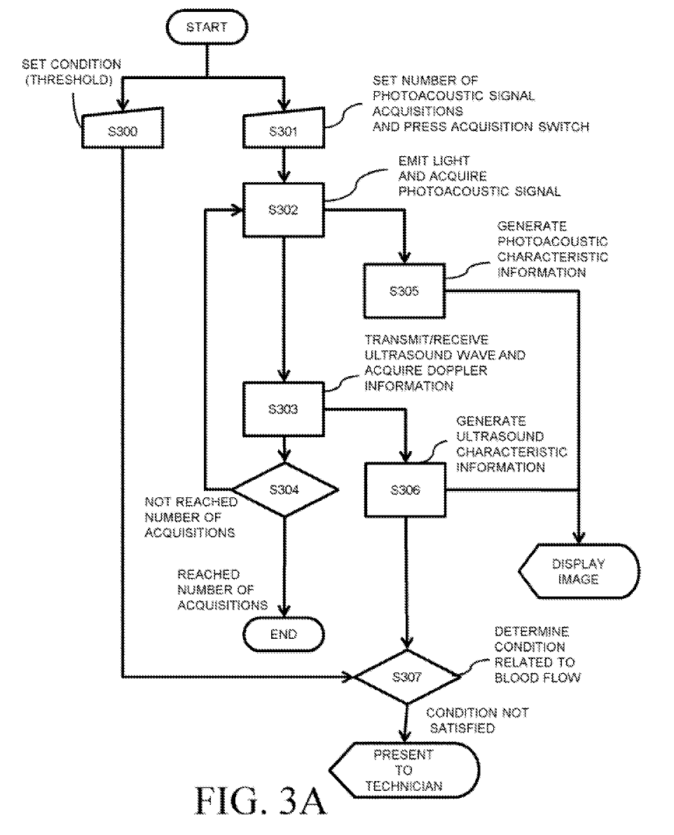

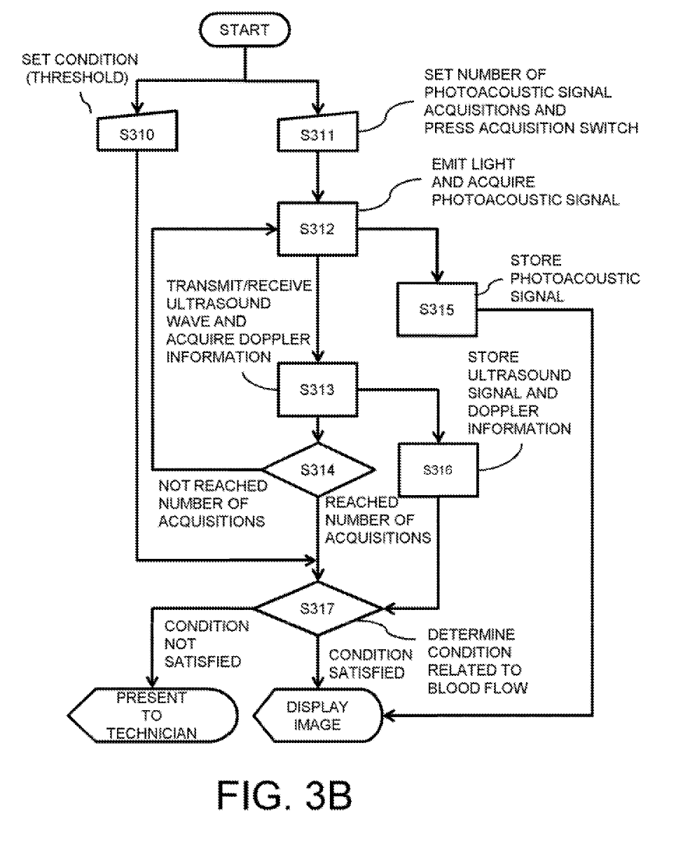

Next, an example of a more detailed process flow will be described with reference to FIGS. 3A and 3B. FIG. 3A shows steps of presenting a PA image and/or a Doppler image obtaining image in real time.

Step S300: After startup of the apparatus, the technician inputs a condition related to blood flow using the inputter. A ROI may be set at the same time.

Step S301: Until the obtaining switch 9 is pressed, the apparatus performs ultrasound wave transmission/reception in the ultrasound measurement mode. In the present step, the technician sets the number of obtaining of a photoacoustic signal using the inputter and operates the obtaining switch 9.

Step S302: The light source 1 irradiates an object with illuminating light. The photoacoustic probe 4 converts a generated photoacoustic wave into a photoacoustic signal.

Step S303: When one photoacoustic signal obtaining is finished, the transmitter/receiver 6 transmits/receives an ultrasound wave and the processing apparatus 7 obtains Doppler information from an ultrasound signal.

Step S304: When the number of photoacoustic signal obtaining set in S301 has not been reached, a return is made to step S302. When the number is reached, the process is ended. When performing mechanical scanning or when using a hand-held probe, S302 to S304 are repeated while moving the probe.

Step S305: The processing apparatus 7 generates image data indicating photoacoustic characteristic information from the photoacoustic signal obtained in S302 and causes the display apparatus 8 to display the image data. When repetitively executing steps S302 to S304, image data may be generated and displayed every time step S302 is performed or image data may be generated and displayed by integrating photoacoustic signals obtained by performing step S302 a plurality of times.

Step S306: The processing apparatus 7 generates image data indicating ultrasound characteristic information from the ultrasound signal obtained in S303 and causes the display apparatus 8 to display the image data. In addition, the processing apparatus 7 may cause the display apparatus 8 to display Doppler information. In the example of the present flow, images are displayed in real time as the photoacoustic signal and the ultrasound signal are obtained.

Step S307: A determination is made on whether or not the Doppler information obtained in S306 satisfies the condition related to blood flow set in S300. When the condition is not satisfied, information to that effect is presented through the presenter 11. When the condition is satisfied, processes of S302 to S304 are continued.

Moreover, when the number of obtaining is reached in S304 and the process is ended, it is also favorable to generate and display a high-definition image using a larger number of signals than the real-time images obtained in S305 and S306. The photoacoustic image, the ultrasound image, and the Doppler information may be arbitrarily combined and displayed in a desired method such as superposition display.

### Another Process Flow

FIG. 3B shows steps of presenting a PA image and/or a Doppler image obtaining image after obtaining of all data is finished.

Steps S310 to S314 are similar to S300 to S304 in FIG. 3A. In these steps, a condition for information related to blood flow is set, the number of signal obtaining is set, the obtaining switch is pressed, and a photoacoustic signal and an ultrasound echo signal are obtained.

Step S315: The storage unit of the processing apparatus 7 stores the photoacoustic signal obtained in S312.

Step S316: The storage unit of the processing apparatus 7 at least stores the Doppler information obtained in S313. An ultrasound signal may be further stored.

Step S317: The threshold set in S310 and the Doppler information stored in S316 are compared with each other to determine whether or not the Doppler information satisfies the condition related to blood flow. When the condition is not satisfied, information to the effect that blood flow is insufficient is presented. On the other hand, when the condition is satisfied, image data indicating photoacoustic characteristic information is generated and displayed. In addition, images indicating ultrasound characteristic information and Doppler information may be generated and displayed.

According to the respective process flows described above, when information obtained by the Doppler method does not satisfy a prescribed condition, a presentation is made to the technician. Subsequently, the technician can adjust an amount by which the photoacoustic probe 4 is pushed into the object. As a result, an effect of a decline in a blood flow rate in photoacoustic measurement is suppressed. In addition, accuracy also improves when measuring oxygen saturation using light with a plurality of wavelengths. Although details will be described later, methods of presentation other than that using the presenter 11 may be adopted. Examples of conceivable methods include generating a photoacoustic image by removing photoacoustic signals obtained before and after a timing at which a prescribed blood flow is not satisfied, stopping emission of light, and suspending or stopping obtaining of photoacoustic signals. Accordingly, unnecessary obtaining of photoacoustic signals and unnecessary generation of photoacoustic characteristic information can be suppressed. Furthermore, even in an apparatus in which the photoacoustic probe 4 performs mechanical scanning, when the prescribed condition is not satisfied, the controller can perform the control described above in a similar manner.

### Second Embodiment

In the present embodiment, when Doppler information does not satisfy a prescribed condition (when a blood flow rate or blood flow speed is insufficient), the controller 10 stops at least one of light emission and photoacoustic signal obtaining. While such control is mainly preferable when displaying real-time images, the control is also applicable when displaying an image after acquiring all data.

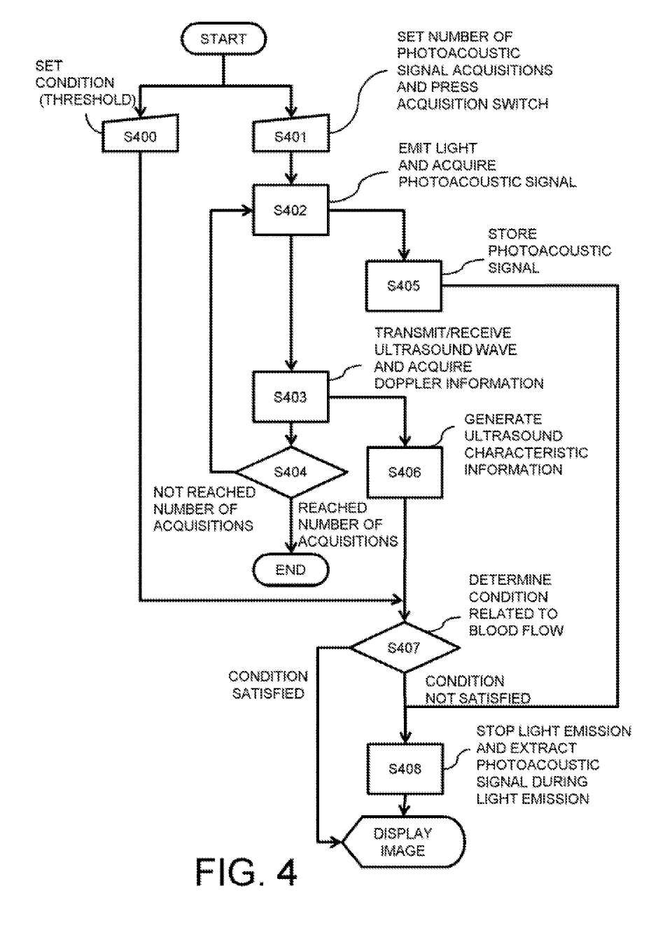

A flow of the present embodiment will be described with reference to FIG. 4 by focusing on portions which differ from the embodiment described above. In step S400, a condition related to blood flow is set. In step S401, the number of photoacoustic measurement is set and the obtaining switch is operated. In steps S402 to S405, light is irradiated as many times as set and signals based on generated photoacoustic waves are stored. In step S406, Doppler information is generated. In step S407, a determination is made on whether or not the obtained Doppler information satisfies the condition related to the prescribed blood flow. When the condition is satisfied, a transition is made to image display. On the other hand, when a determination is made in the present embodiment that the condition is not satisfied, in step S408, the controller 10 stops at least one of light emission and photoacoustic signal obtaining.

After the stoppage, photoacoustic signals obtained up to then may be extracted, and a photoacoustic image may be generated based on the extracted photoacoustic signals and displayed on the display apparatus 8. Alternatively, light emission and photoacoustic signal obtaining may be simply suspended instead of being stopped. In this case, Doppler information may be periodically obtained, and light emission and photoacoustic signal obtaining may be restarted once blood flow is restored to a prescribed level or a higher level.

In the present embodiment, when Doppler information does not satisfy a prescribed condition and a determination is made that blood flow is insufficient, light emission and photoacoustic signal obtaining are suspended or stopped. As a result, a photoacoustic image can be generated from photoacoustic signals obtained when blood flow is sufficient. Therefore, in addition to an improvement in contrast, an effect of not performing unnecessary light emission and unnecessary photoacoustic signal obtaining when blood flow is low is produced.

### Third Embodiment

In the present embodiment, a photoacoustic signal obtained when Doppler information does not satisfy a prescribed condition (when a blood flow rate or blood flow speed is insufficient) is not used to generate photoacoustic characteristic information. Such control is mainly preferable when displaying an image after acquiring all data as shown in FIG. 3B. However, even when displaying a real-time image as shown in FIG. 3A, the control is applicable when generating an image using all ultimately obtained signals.

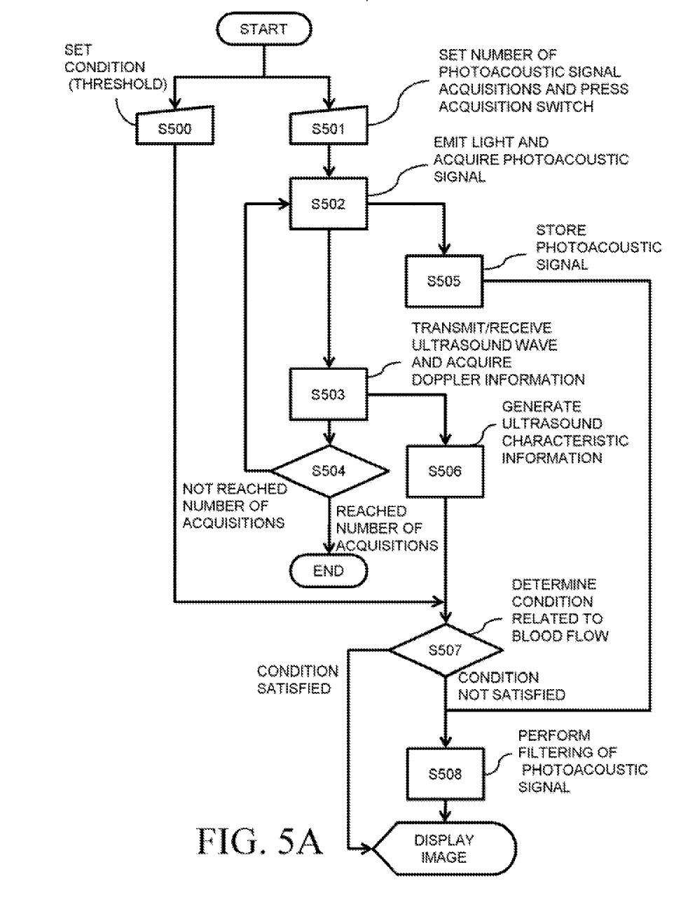

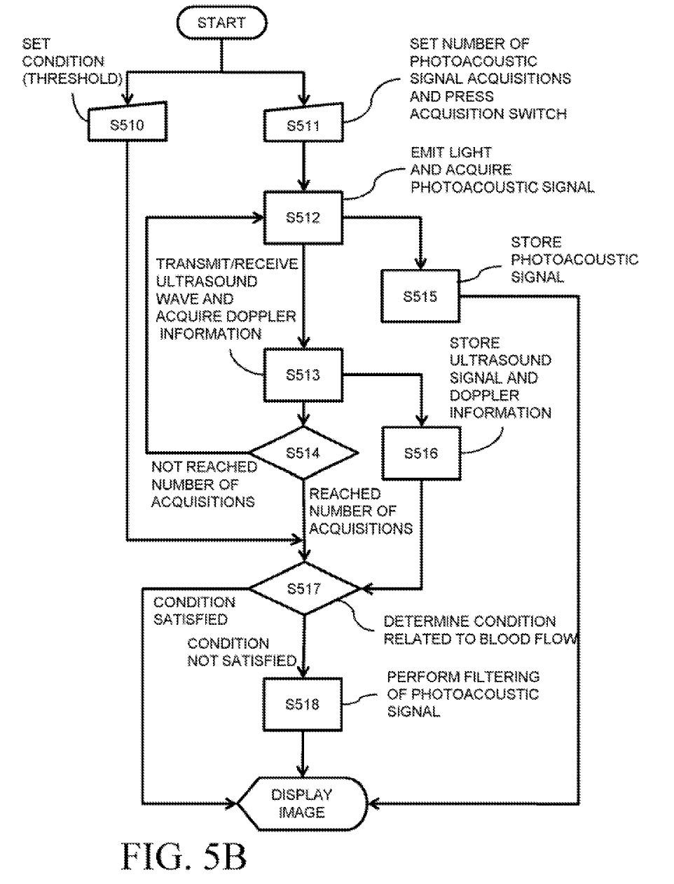

FIG. 5A is a flow chart showing processes of presenting a photoacoustic image and/or a Doppler image in real time. Portions which differ from the second embodiment described above will be mainly described. In step S507, when it is determined that the condition related to blood flow is satisfied, an ultrasound image and/or Doppler information are displayed on the display apparatus 8. On the other hand, when the condition is not satisfied in the present embodiment, in step S508, using the Doppler information stored in S506 as a filter, a photoacoustic signal obtained when the prescribed blood flow is obtained is extracted from the photoacoustic signal stored in S505. In addition, image information is generated from the extracted photoacoustic signal and displayed on the display apparatus 8.

Meanwhile, FIG. 5B is a flow chart showing processes of presenting a photoacoustic image and/or a Doppler image after obtaining of all data is finished. In step S517, a determination is made on whether or not each piece of Doppler information at the time of obtaining of each photoacoustic signal satisfies the condition related to the prescribed blood flow. The processing apparatus 7 extracts a photoacoustic signal obtained when the prescribed blood flow is obtained, and generates photoacoustic characteristic information using the extracted photoacoustic signal and causes the display apparatus 8 to display the photoacoustic characteristic information. Typically, only photoacoustic signals before and after a timing of transmission/reception of an ultrasound wave from which Doppler information not satisfying the condition had been obtained are excluded from use. However, an extraction method is not limited to this method. For example, a method may be adopted of not using photoacoustic signals obtained within a prescribed period of time from a timing of transmission/reception of an ultrasound wave from which Doppler information not satisfying the condition had been obtained.

According to the present embodiment, a photoacoustic signal obtained when a blood flow rate is insufficient is not used to obtain photoacoustic characteristic information. As a result, since a photoacoustic image is generated from photoacoustic signals obtained when blood flow is sufficient, contrast is improved.

In the first to third embodiments, the controller 10 controls a light source, a signal processing circuit, and the like based on the number of photoacoustic signal obtaining set in advance. However, a control method is not limited thereto. For example, a stop switch may be provided on a case of a probe or the like separately from the obtaining switch 9. Alternatively, a stop instruction may be received via the inputter of the information processing apparatus.

### Fourth Embodiment

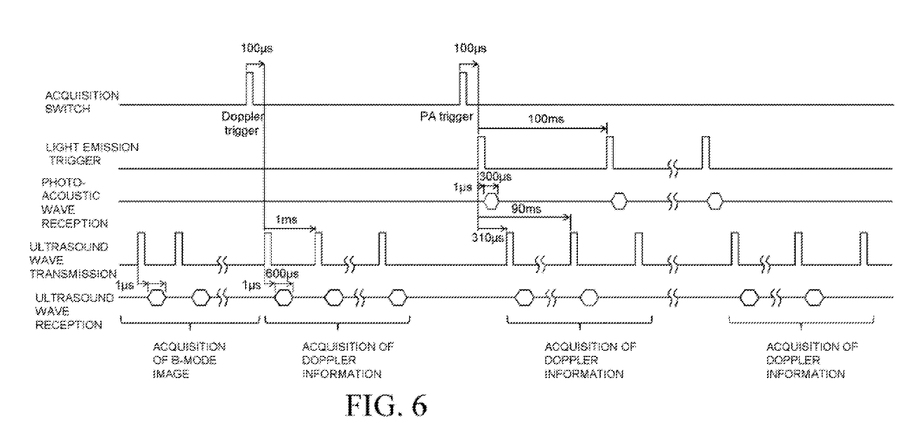

In the present embodiment, Doppler information is obtained prior to photoacoustic signal obtaining. FIG. 6 is a timing chart illustrating the present embodiment. When the obtaining switch 9 is pressed, the obtaining switch 9 first acts as a trigger (a Doppler trigger) for Doppler information obtaining and ultrasound wave transmission/reception is performed. In addition, Doppler information is obtained from a received signal thereof and displayed on the display apparatus 8.

The technician checks blood flow based on the Doppler information. When it is determined that sufficient blood flow is obtained, by once again pressing the obtaining switch 9, the obtaining switch 9 acts as a trigger (PA trigger) for photoacoustic signal obtaining. Accordingly, light emission for photoacoustic obtaining and photoacoustic signal obtaining are performed. On the other hand, when it is determined that the prescribed blood flow is not obtained, photoacoustic signal obtaining is not performed. Alternatively, the processing apparatus 7 may perform the determination of Doppler information. When the processing apparatus 7 determines that sufficient blood flow is not obtained, the controller 10 may perform processes such as in the first to third embodiments or may suppress a next reception of the obtaining switch 9.

According to the present embodiment, the technician can be prompted to check blood flow in advance. In addition, when it is determined that sufficient blood flow is not obtained, for example, the technician reduces a force by which the photoacoustic probe 4 is pressed against an object. As a result, a photoacoustic signal can be obtained in a state where blood flow is obtained and contrast of a photoacoustic image is improved. Furthermore, accuracy also improves when measuring oxygen saturation using light with a plurality of wavelengths.

Moreover, in FIG. 6, ultrasound waves are transmitted/received between a photoacoustic signal obtaining and a next photoacoustic signal obtaining. Based on a received signal thereof, information to the effect that blood flow is low may be presented (first embodiment), light emission and photoacoustic signal obtaining may be stopped or suspended (second embodiment), or a signal during generation of photoacoustic characteristic information may be extracted (third embodiment).

As described above, according to the present invention, a determination of a state of blood flow based on Doppler information can be made in photoacoustic measurement. As a result, since a photoacoustic image in a state where blood flow inside a living organism is secured can be obtained, an effect of a decline in a blood flow rate in photoacoustic measurement can be suppressed.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-084736, filed on Apr. 20, 2016, which is hereby incorporated by reference herein in its entirety.

What is claimed is:

1. An object information obtaining apparatus, comprising:
an irradiator configured to irradiate an object with light output from a light source;
a transmitting element configured to transmit a transmission ultrasound wave;
a receiving element configured to receive a reception acoustic wave and output an electrical signal;
a signal processor;
an information processor; and
a controller, wherein
the signal processor is configured to generate a photoacoustic signal from the electrical signal derived from a photoacoustic wave generated inside the object when the object is irradiated with the light and to generate an ultrasound echo signal from the electrical signal derived from an echo wave generated when the transmission ultrasound wave is reflected inside the object,
the information processor is configured to obtain photoacoustic characteristic information of the object using the photoacoustic signal and to obtain ultrasound characteristic information of the object including information related to blood flow of the object using the ultrasound echo signal, and
the controller is configured to, in accordance with the information related to the blood flow, perform control of at least one of irradiation of the light, generation of the photoacoustic signal, and obtaining of the photoacoustic characteristic information.

2. The object information obtaining apparatus according to claim 1, wherein
the controller is configured to perform the control in accordance with whether or not the information related to the blood flow satisfies a prescribed condition.

3. The object information obtaining apparatus according to claim 2, wherein
the prescribed condition is whether or not a blood flow rate or a blood flow speed of the object exceeds a threshold.

4. The object information obtaining apparatus according to claim 2, wherein
the prescribed condition is whether or not a value related to an amount of decline of the blood flow rate or the blood flow speed exceeds a threshold.

5. The object information obtaining apparatus according to claim 2,
further comprising a presenter, wherein
the controller is configured to, when the prescribed condition is not satisfied, present through the presenter information to the effect that the prescribed condition is not satisfied.

6. The object information obtaining apparatus according to claim 2, wherein
the controller is configured to, when the prescribed condition is not satisfied, stop the irradiation of the light, the generation of the photoacoustic signal, or the obtaining of the photoacoustic characteristic information.

7. The object information obtaining apparatus according to claim 2, wherein
the controller is configured to, when the prescribed condition is not satisfied, suspend the irradiation of the light, the generation of the photoacoustic signal, or the obtaining of the photoacoustic characteristic information.

8. The object information obtaining apparatus according to claim 7, wherein
the controller is configured to, when the prescribed condition is satisfied after suspending the irradiation of the light, the generation of the photoacoustic signal, or the obtaining of the photoacoustic characteristic information, restart the irradiation of the light, the generation of the photoacoustic signal, or the obtaining of the photoacoustic characteristic information.

9. The object information obtaining apparatus according to claim 2, wherein
the information processor is configured not to, when obtaining the photoacoustic characteristic information, use the photoacoustic signal obtained when the prescribed condition is not satisfied.

10. The object information obtaining apparatus according to claim 1,
further comprising an inputter, wherein
the controller is configured to, in accordance with an operation performed by a user via the inputter, switch the object information obtaining apparatus to a photoacoustic measurement mode of obtaining the photoacoustic characteristic information or to an ultrasound echo mode of obtaining the ultrasound characteristic information.

11. The object information obtaining apparatus according to claim 1,
further comprising an inputter, wherein
the controller is configured to receive designation of a region of interest, for which the ultrasound characteristic information is to be obtained, from the user via the inputter.

12. The object information obtaining apparatus according to claim 10, wherein
the controller is configured to, when switching to the photoacoustic measurement mode, determine whether or not the information related to the blood flow satisfies a prescribed condition.

13. The object information obtaining apparatus according to claim 10,
further comprising a case including the transmitting element, the receiving element, and an exit end of the light, wherein
the inputter is a switch provided on the case.

14. A control method of an object information obtaining apparatus including an irradiator which irradiates an object with light output from a light source, a transmitting element which transmits a transmission ultrasound wave, a receiving element which receives a reception acoustic wave and which outputs an electrical signal, a signal processor, an information processor, and a controller, wherein the control method of an object information obtaining apparatus comprises:
causing the signal processor to generate a photoacoustic signal from the electrical signal derived from a photoacoustic wave generated inside the object when the object is irradiated with the light and generate an ultrasound echo signal from the electrical signal derived from an echo wave generated when the transmission ultrasound wave is reflected inside the object;
causing the information processor to obtain photoacoustic characteristic information of the object using the photoacoustic signal and obtain ultrasound characteristic information of the object including information related to blood flow of the object using the ultrasound echo signal; and
causing the controller to perform control of, in accordance with the information related to the blood flow, at least one of irradiation of the light, generation of the photoacoustic signal, and obtaining of the photoacoustic characteristic information.

15. The control method of an object information obtaining apparatus according to claim 14, wherein
the controller is caused to perform the control in accordance with whether or not the information related to the blood flow satisfies a prescribed condition.

16. The control method of an object information obtaining apparatus according to claim 15, wherein
the prescribed condition is whether or not a blood flow rate or a blood flow speed of the object exceeds a threshold.

17. The control method of an object information obtaining apparatus according to claim 15, wherein
the prescribed condition is whether or not a value related to an amount of decline of the blood flow rate or the blood flow speed exceeds a threshold.
